(19) United States
(12) Patent Application Publication
(10) Pub. No.: US 2008/0299552 A1
(43) Pub. Date: Dec. 4, 2008

(54) Method For Determining The Function Of Nucleic Acid Sequences And Expression Products Coded Thereby

(75) Inventors: Ugur Sahin, Mainz (DE); Ozlem Tureci, Mainz (DE); Carolin Bell, Guldental (DE)

Correspondence Address: Olson & Cepuritis, LTD., 20 NORTH WACKER DRIVE, 36TH FLOOR, CHICAGO, IL, 60606, US

(73) Assignee: Johannes Gutenberg-Universitat Mainz Vertreten Durch Den Prasidenten, Mainz (DE)

(21) Appl. No.: 11/794,367

(22) PCT Filed: Dec. 22, 2005
(86) PCT No.: PCT/EP2005/013920

§ 371 Date: Apr. 28, 2008

(30) Foreign Application Priority Data
Dec. 29, 2004 (DE) 10 2004 063 265.0

(51) Int. Cl. C12Q 1/68

(52) U.S. Cl. 435/6

(57) ABSTRACT

The invention relates to a method for the investigation and determination of the function of nucleic acid sequences and nucleic acid expression products by the introduction of RNA into host cells.

### FIELD OF THE INVENTION

The invention relates generally to a method for investigation and determination of the function of nucleic acid sequences and nucleic acid expression products by introducing RNA into host cells.

### BACKGROUND OF THE INVENTION

With the completion of the first draft of the sequencing of the human genome, the next challenge is to progress from descriptive genome research to functional gene research (Fields, S. et al. (1999) Proc. Natl. Acad. Sci. U.S.A. 96, 8825-8826).

The use of antisense-RNA or RNAi (RNA interference) has found wide application in recent years for the targeted switching-off of expression of a gene.

Antisense-RNA binds sequence-specifically, according to the rules of complementarity, to the naturally transcribed mRNA of its target sequence. This method was first described in prokaryotes (Green, P. J. et al. (1986) Annu. Rev. Biochem. 55, 569-597). It now permits rapid, transient, post-transcriptional silencing of any desired gene in a great many types of eukaryotic cells (Izant, J. G. and Weintraub, H. (1985) Science 229, 345-352) and is widely used.

In RNAi (RNA interference), short (˜21 bp) double-stranded RNA that interacts specifically with the target sequence is introduced into cells. As a result, a multiprotein-complex is activated, which leads to premature degradation of transcribed mRNA before the latter can be translated (Hammond, S. M. et al. (2000) Nature 404, 293-296). However, siRNA can also cause nonspecific effects through activation of the interferon system, through induction of the protein kinase response (PKR), which is dependent on double-stranded RNA (Elbashir, S. M. et al. (2001) Nature 411, 494-498; Caplen, N. J. et al. (2001) Proc. Natl. Acad. Sci. U.S.A. 98, 9742-9747) or through concentration-dependent stimulation or repression of nontargeted genes (Persengiev, S. P. et al. (2004) RNA 10, 12-18).

The methods described above, using antisense-RNA or RNAi, prevent translation and therefore lead to a loss of function of genes.

However, for a functional characterization of particular genes, especially genes that are activated or overexpressed ectopically, such as genes expressed selectively in tumors, it is more useful to simulate the effects of a gain of function.

A number of techniques of gene transfer are also available for this. Thus, both transient transfectants and stably transfected cellular clones can be produced. The gene under investigation can be inserted in cells by means of DNA plasmid constructs or with the aid of retroviral or adenoviral vectors. Moreover, expression of the inserted gene can be constitutive or can be controlled via a controllable promoter. All forms of gene transfer used for this purpose have system-inherent drawbacks. Transient transfections with plasmids or viruses are very inefficient with most cell types (especially primary cells) and result in the transfection of variable subpopulations, which moreover does not allow sufficiently accurate titration of the level of expression. However, since one and the same gene product can perform variable functions in different cells or even in the same cells at different concentration, the amount of information provided by these techniques is very limited. Another problem that should not be underestimated is that transfections with plasmids only lead to transcription after a delay. The time until transcription is dependent on the phase of the cell cycle in which the cells were transfected, and is accordingly very variable in cell populations. The consequence is unsynchronized transcription and expression. An alternative is offered by stably transfected cells. However, the production of stably transfected cell lines is very time-consuming and cannot be performed efficiently for all cell systems. Thus, most freshly isolated cells of a mammalian organism cannot be transfected stably. Another disadvantage is that the enrichment of transfected cells after transfection, e.g. by the use of antibiotics or by physical measures (e.g. cell sorting) leads to the selection of subpopulations which differ in many respects from the original cell type, regardless of the gene product introduced.

The present invention relates to a method for the functional characterization of gene products, which is based on the transfer of in vitro transcribed, long-chain RNA. Transfer of in vitro transcribed RNA into cells is an established method and is currently being tested particularly intensively in tumor immunology, for introducing tumor antigens into antigen-presenting dendritic cells (DC) (Boczkowski, D. et al. (2000) Cancer Res. 60, 1028-1034; Heiser, A. et al. (2001) J. Immunol. 166, 2953-2960; Nair, S. K. et al. (1998) Nat. Biotechnol. 16, 364-369). In contrast, the transfer of in vitro transcribed RNA has not been used so far for the systematic determination of gene function.

### SUMMARY OF THE INVENTION

The invention relates generally to methods for the functional characterization of nucleic acids and/or expression products, especially peptides and proteins. In the methods according to the invention, the function of the nucleic acid under investigation and/or of the expression product under investigation can be entirely unknown or one or more functions of the nucleic acid under investigation and/or expression product under investigation can be known, and in this embodiment the methods according to the invention aim to identify additional functions.

One aspect of the invention relates to a method for investigating the effects of the expression of a nucleic acid in a host cell, comprising the steps: (a) introduction of RNA, derived from the nucleic acid, into the host cell; and (b) investigation of effects on the host cell, arising as a result of introduction of the RNA into the host cell.

The effects on host cell, which arise as a result of introduction of the RNA into the host cell, can provide information about the function of the nucleic acid and/or of the expression product.

Thus, another aspect of the invention relates to a method for investigating the function of a nucleic acid and/or of a nucleic acid expression product in an organism, comprising the steps: (a) introduction of RNA that has been derived from the nucleic acid and/or codes for a portion or the whole sequence of the expression product, into a host cell; (b) investigation of effects on the host cell, resulting from introduction of the RNA into the host cell; and (c) identification of one or more functions of the nucleic acid and/or of the expression product on the basis of the effects on the host cell determined in step (b).

The methods according to the invention have advantages in particular with respect to little expenditure of time, the possibility of analysis of kinetics, and applicability in almost all cell systems. In particular, introduction of the nucleic acid under investigation, e.g. of a gene, in the form of RNA has the advantage, relative to the use of DNA constructs, that for expression, RNA need only get into the cytoplasm of the cells, not into the cell nucleus. Therefore RNA transfer is not dependent on the division activity of the cells to be transfected. Furthermore, the transfection rates attainable with RNA are relatively high (Van Tendeloo, V. F. et al. (2001) Blood 98, 49-56; Saeboe-Larssen, S. et al. (2002) J. Immunol. Methods 259, 191-203) and the amounts of protein achieved correspond to those in physiological expression. Kinetic studies can be conducted very well, because protein that has already been translated is detectable within a few hours and becomes functionally relevant (Van Meirvenne, S. et al. (2002) Cancer Gene Ther. 9, 787-797).

The transfer of RNA into cells is, according to the invention, ideal for subsequent functional characterization on the basis of the following characteristics, demonstrated with examples:
RNA transfer permits gene products to be inserted in all eukaryotic cell types investigated, including tumor cells and untransformed primary cells (see Table 1)
Transfection rates are very high, for many cell types even >90%, therefore there is no need for selection.
The level of expression can be titrated very well by the amount of RNA used for transfection. In this way it is possible to simulate the effects of different levels of expression in the cells.
After transfection, synchronized translation takes place immediately, permitting sensitive detection of biological after-effects in chronological order.
RNA transfection displays little method-dependent variation of the transfected cells with respect to their transcriptome.

The methods according to the invention are suitable in particular for the simulation of ectopic expression of transcription factors, oncogenes or potential oncogenes in cells and cell lines and therefore for functional characterization of such genes. In these cases the RNA that is inserted into the host cells is derived from an oncogene or potential oncogene.

In one embodiment of the methods according to the invention, the RNA that is to be inserted into a host cell is obtained by cloning of the nucleic acid that encodes it and transcription of the cloned nucleic acid, preferably by in vitro transcription. The promoter for controlling transcription can be any promoter for an RNA polymerase. Particular examples of RNA polymerases are the T7, T3 and SP6 RNA polymerases. Preferably the in vitro transcription according to the invention is controlled by a T7 or SP6 promoter.

In particular embodiments, the nucleic acid coding for the RNA is present in conjunction with a polyadenyl cassette. In these embodiments the RNA obtained by expression of the nucleic acid, preferably by in vitro transcription, preferably has a polyadenylation of a defined length. Preferably the poly-A stretch has a length of 10 to 500, more preferably 30 to 300, even more preferably 65 to 200 and especially 100 to 150 adenosine residues. In an especially preferred embodiment the poly-A stretch has a length of approximately 120 adenosine residues.

In further embodiments of the invention, the RNA that is to be inserted into a host cell has, at its 5′ end, a Cap structure or a regulatory sequence, which promotes the translation in the host cell.

In especially preferred embodiments of the methods according to the invention, introduction of the RNA into the host cell causes expression of the expression product encoded by the RNA. In particular embodiments of the invention, the expression product can be selected from the group consisting of peptides, proteins, enzymes, antibodies and antigens.

According to the invention, for functional investigations it is possible to control the amount of RNA that is introduced into a host cell, and hence the amount of the expression product expressed in the host cell.

In particular embodiments, the RNA that is to be introduced into a host cell according to the invention is a portion of an RNA library. This aspect of the invention also relates to a method for screening a group of different nucleic acids or variants of a nucleic acid (Def.: RNA library) for the occurrence of RNA which, on introduction into a host cell, is capable of producing a particular effect on the host cell or of controlling or influencing a particular function of the host cell.

This aspect of the invention therefore relates to a method of finding a nucleic acid and/or a nucleic acid expression product with a particular effect/function, comprising the steps: (a) introduction of RNA into a host cell; (b) investigation of effects on the host cell, arising as a result of introduction of the RNA into the host cell; and (c) identification of the RNA that causes the effects on the host cell. This method can also comprise determination of the nucleotide sequence of the RNA that causes the effects on the host cell. Said determination of the nucleotide sequence can take place in a known manner, in particular by sequencing of the original nucleic acid.

The methods according to the invention comprise an investigation and identification of effects on a host cell, that arise as a result of introduction of RNA into the host cell and possibly expression of the RNA in the host cell.

It will be clear to a person skilled in the art that there are many possible ways in which the introduction of RNA into a host cell can have effects on this host cell. Accordingly, there are various methods by which effects that arise as a result of the introduction of RNA into a host cell can be investigated in the methods according to the invention. Such methods comprise, in a non-limiting manner, the determination of one or more of the following parameters: cell proliferation, cell death, cell migration, cell adhesion, altered resistance or altered sensitivity to drugs, aging of cells, production or inhibition of the production of particular molecules, amounts of mRNA, amounts of protein expression, protein activity levels, effects on protein phosphorylation, effects on processing of protein or nucleic acid, effects on RNA stability, effects on signal transduction or second messengers.

Preferably the investigation of effects on a host cell, which arise as a result of introduction of RNA into the host cell, comprises an investigation of phenotypic and/or biochemical characteristics of the host cell.

In particular embodiments, the investigation of effects on a host cell, which arise as a result of the introduction of RNA into the host cell, comprises an investigation of the protein expression profile (proteome analysis) and/or an investigation of the RNA expression profile (transcriptome analysis) of the host cell.

In further embodiments, the investigation of effects on a host cell, which arise as a result of introduction of RNA into the host cell, comprises an investigation of the metabolism of the host cell. Preferably an investigation of the metabolism comprises an investigation of the accumulation of substrates or products from enzymatic reactions according to known methods.

In other embodiments, the investigation of effects on a host cell, which arise as a result of introduction of RNA into the host cell, comprises an investigation of gene regulation.

In an especially preferred embodiment of the methods according to the invention, the investigation of effects on a host cell is carried out in comparison with a cell into which the RNA was not introduced (transfer control) or in comparison with a cell into which an RNA that is irrelevant to the function (negative control) or an RNA that mediates the function (positive control) was introduced. These cells serve as controls or as references. These cells preferably correspond to the host cell that is used.

A subtractive hybridization can be used in order to determine whether the introduction of RNA into a host cell comprises an effect on the RNA expression profile of the host cell. In this embodiment, for example RNA present in control cells is “subtracted” from RNA that is expressed by host cells, into which RNA of interest was inserted.

In a further embodiment, the investigation of effects on a host cell, which arise as a result of the introduction of RNA into the host cell, comprises a complementation analysis. In this embodiment, the function of a gene endogenously present in the host cell is replaced or intensified by introduction of RNA of interest into the host cell.

In a further embodiment, the investigation of effects on a host cell, which arise as a result of introduction of RNA into the host cell, comprises an investigation of phenotypic characteristics of the host cell, e.g. by methods that include a morphological, macroscopic or microscopic investigation.

The host cells can also undergo a further treatment or change of conditions before an investigation of effects on a host cell, which arise as a result of the introduction of RNA into the host cell. Thus, in one embodiment, effects on cellular factors are investigated temporarily. The host cells can also be integrated into an in vivo context, in particular an organism, for further determination of a function of a nucleic acid and/or of a nucleic acid expression product.

The investigation of effects on a host cell, which arise as a result of the introduction of RNA into the host cell, makes it possible to identify one or more functions that a nucleic acid and/or an expression product has in an organism, from which this nucleic acid and/or the expression product is derived.

The methods according to the invention also make it possible to identify one or more cellular factors that are functionally related to the nucleic acid of interest and/or the expression product of interest. Such a group of cellular factors would display increased expression on introduction of the RNA of interest into a host cell. Another such group of cellular factors would display decreased expression on introduction of the RNA of interest into a host cell. The invention thus also provides means for the identification of functional relations between cellular factors and nucleic acids and/or expression products of interest.

In certain embodiments it is preferable for the host cell to be derived from the same organism as the nucleic acid that is to be investigated and/or the expression product that is to be investigated, i.e. the nucleic acid that is to be investigated and/or the expression product that is to be investigated is homologous with the host cell. However, it is possible for the nucleic acid that is to be investigated and/or the expression product that is to be investigated to be heterologous to the host cell.

Preferably the nucleic acid of interest and/or the expression product of interest is at least initially investigated in a host cell that is known to express the nucleic acid and/or the expression product naturally.

In certain embodiments, however, it may be desirable to test heterologous nucleic acids and/or expression products in host cells, especially mammalian cells. Thus, it is possible to determine, for example, whether a particular sequence, e.g. from a fungus, is capable of functioning as a substitution product for a mammalian sequence. If this is so, the heterologous sequence can for example be used as a therapeutic substitution product for the mammalian sequence.

Furthermore, it may be desirable to test derivatives of a nucleic acid and/or of an expression product for one or more functions in the methods according to the invention. This makes it possible to alter a particular nucleic acid and/or a particular expression product and to test whether the altered nucleic acid and/or the altered expression product retains one or more effects and/or functions of the original nucleic acid and/or of the original expression product. In many cases it may be desirable to produce derivatives of a nucleic acid and/or of an expression product that retain one or more effects and/or functions of the original nucleic acid and/or of the original expression product, whereas they do not have one or more effects and/or functions of the original nucleic acid and/or of the original expression product.

In certain embodiments of the invention, high throughput systems are used for carrying out the methods according to the invention. In one embodiment of this aspect, the system can optionally be computerized or roboticized and can also comprise the use of a large number of compartments. In an especially preferred embodiment, the compartments are arranged in a multiwell plate and the RNA and the host cells are preferably arranged in a large number of compartments.

Effects on a host cell, which arise as a result of the introduction of RNA into the host cell, can be investigated by means of microarray techniques and arrangements that are known.

### Precise Description of the Invention

According to the invention, standard methods can be used for production of recombinant nucleic acids, cultivation of cells and introduction of nucleic acids, in particular RNA, into cells, in particular electroporation and lipofection. Enzymatic reactions and purification techniques are carried out according to the manufacturers' instructions or in a known manner.

The term “organism” relates according to the invention to any biological unit that is capable of multiplying or transmitting genetic material and comprises plants and animals, and microorganisms such as bacteria, yeasts, fungi and viruses.

The term “transfection” relates according to the invention to the introduction of one or more nucleic acids into an organism or into a cell or cells derived from the latter.

The term “host cell” relates according to the invention to any cell that can be transformed or transfected with an exogenous nucleic acid, in particular RNA. A nucleic acid can be present in the host cell in one or more copies and is, in one embodiment, expressed in the host cell.

The term “host cells” comprises, according to the invention, prokaryotic and eukaryotic cells, in particular human and animal cells, plant cells and cells of microorganisms. Especially preferred host cells used according to the invention are eukaryotic cells, in particular mammalian cells such as cells from human, mouse, hamster, pig, goat and primates. The cells can be derived from a great variety of tissue types and comprise primary cells and cell lines.

Preferred host cells are those from human tissues, including but not limited to nerve cells, brain cells, epithelial cells, connective tissue cells (such as fibroblasts and osteoblasts), blood cells (such as leukocytes, lymphocytes, monocytes and neutrophils, in particular dendritic cells, thrombocytes), sensory cells, muscle cells, lung cells, heart cells, liver cells, skin cells, pancreas cells, mammary cells, kidney cells, intestinal cells, gastric cells, colon cells, prostate cells, ovarian cells, germ cells and stem cells, which have the capacity to differentiate into various cell types.

Cultivated cell lines can also be used. In certain embodiments of the invention, partially or fully differentiated cells are used, for example if it is known that the nucleic acid of interest and/or the expression product of interest is normally only expressed in these cells.

The term “effects on a host cell, which arise as a result of the introduction of RNA into the host cell” comprises, according to the invention, any perceptible effects on a host cell that can be attributed to the introduction of the RNA into the host cell, in particular effects on the growth, the development, the multiplication, the hereditary transmission, the biochemistry and/or the phenotype of the host cell.

The term “phenotype” or “phenotypic characteristic” relates to the appearance of an organism or of a host cell. The phenotype includes all internal and external structures and functions. In the course of individual development, the phenotype of an organism and of the cells derived from it can change. The external characteristics of an organism and of the cells derived from it are determined by its genetic information (genotype), but the phenotype depends on which genes are actually expressed (expression).

The term “biochemistry” or “biochemical characteristic” relates to metabolism and the transport of substances in an organism or in a cell derived from it.

The term “function of a nucleic acid and/or of a nucleic acid expression product in an organism or the cells derived from it” relates, according to the invention, to the functional role of the nucleic acid and/or the nucleic acid expression product in an organism or in cells derived from it, preferably in the organism or in the cells derived from it, from which the nucleic acid and/or the nucleic acid expression product was derived. In one embodiment the term relates to the functional role of a nucleic acid and/or of a nucleic acid expression product in carcinogenesis. With reference to a nucleic acid, the term relates in particular to the effects resulting from an expression or lack of expression of the nucleic acid in the organism or in the cells derived from it.

The term “function of a nucleic acid and/or of a nucleic acid expression product” can mean, according to the invention, that the nucleic acid and/or the nucleic acid expression product has only one function or has several functions, and in the latter case one or more functions are investigated by the methods according to the invention.

The term “gene” relates according to the invention to a particular nucleic acid sequence, which is responsible for the production of one or more cellular products and/or for the attainment of one or more intercellular or intracellular functions. In particular the term relates to a DNA segment that codes for a specific protein or a functional or structural RNA molecule.

A nucleic acid according to the invention is preferably ribonucleic acid (RNA) or deoxyribonucleic acid (DNA), which can be used for the production of RNA. Nucleic acids comprise, according to the invention, genomic DNA, cDNA, mRNA, molecules produced by recombinant technology and those synthesized chemically. A nucleic acid can, according to the invention, be in the form of a single-stranded or double-stranded and linear or covalently circularly closed molecule.

A nucleic acid, in particular RNA, according to the invention is derived from another nucleic acid if there is a certain degree of homology between the nucleic acids, i.e. the nucleic acids have significant direct or complementary matches in the sequence of their nucleotides. Moreover, a nucleic acid is also derived from another nucleic acid if the first nucleic acid is a derivative of the second nucleic acid. The term “RNA that has been derived from a nucleic acid” means in particular that the RNA is a transcription product of the nucleic acid or of a nucleic acid derived from it and/or has a sequence that results on translation in a protein expression product that has the same sequence as a protein expression product that arises on the basis of transcription and translation of the nucleic acid from which the RNA was derived.

“Derivative” of a nucleic acid means, according to the invention, that there are single or multiple nucleotide substitutions, deletions and/or additions in the nucleic acid. The term “derivative” also further comprises a chemical derivatization of a nucleic acid on a nucleotide base, on the sugar or on the phosphate. The term “derivative” also comprises nucleic acids that contain non-naturally occurring nucleotides and nucleotide analogs.

A nucleic acid can, according to the invention, be isolated. The term “isolated nucleic acid” means, according to the invention, that the nucleic acid (i) was amplified in vitro, for example by a polymerase chain reaction (PCR), (ii) was produced recombinantly by cloning, (iii) was purified, for example by cleavage and separation by gel electrophoresis or (iv) was synthesized, for example by chemical synthesis. An isolated nucleic acid is a nucleic acid that is available for manipulation by recombinant DNA techniques.

A nucleic acid is “complementary” to another nucleic acid if the two sequences can hybridize with one another and form a stable duplex, the hybridization preferably taking place under conditions that permit specific hybridization between polynucleotides (stringent conditions). Stringent conditions are described for example in Molecular Cloning: A Laboratory Manual, J. Sambrook et al., Publ., 2nd edition, Cold Spring Harbor Laboratory Press, Cold Spring Harbor, N.Y., 1989 or Current Protocols in Molecular Biology, F. M. Ausubel et al., Publ., John Wiley & Sons, Inc., New York and relate for example to hybridization at 65° C. in hybridization buffer (3.5×SSC, 0.02% Ficoll, 0.02% polyvinylpyrrolidone, 0.02% bovine serum albumin, 2.5 mM NaH₂PO₄ (pH7), 0.5% SDS, 2 mM EDTA). SSC is 0.15 M sodium chloride/0.15 M sodium citrate, pH 7. After hybridization, the membrane onto which the DNA was transferred is washed for example in 2×SSC at room temperature and then in 0.1-0.5×SSC/0.1×SDS at temperatures up to 68° C.

Complementary nucleic acids have, according to the invention, at least 70%, at least 80%, at least 90% and preferably at least 95%, at least 98% or at least 99% identity of the nucleotides.

Expression control sequences or regulatory sequences, which according to the invention are linked functionally with a nucleic acid, can be homologous or heterologous with respect to the nucleic acid. A coding sequence and a regulatory sequence are linked together “functionally” if they are bound together covalently, so that the transcription or translation of the coding sequence is under the control or under the influence of the regulatory sequence. If the coding sequence is to be translated into a functional protein, with functional linkage of a regulatory sequence with the coding sequence, induction of the regulatory sequence leads to a transcription of the coding sequence, without causing a reading frame shift in the coding sequence or inability of the coding sequence to be translated into the desired protein or peptide.

The term “expression control sequence” or “regulatory sequence” comprises, according to the invention, promoters, ribosome-binding sequences and other control elements, which control the transcription of the gene or the translation of the derived RNA. In certain embodiments of the invention, the expression control sequences can be controlled. The precise structure of regulatory sequences can vary depending on the species or depending on the cell type, but generally comprises 5′-untranscribed and 5′ and 3′-untranslated sequences, which are involved in the initiation of transcription or translation, such as TATA-box, capping-sequence, CAAT-sequence and the like. In particular, 5′-untranscribed regulatory sequences comprise a promoter region that includes a promoter sequence for transcriptional control of the functionally bound gene. Regulatory sequences can also comprise enhancer sequences or upstream activator sequences.

Furthermore, a nucleic acid according to the invention can be linked to another nucleic acid which codes for a polypeptide that guides a protein or polypeptide, which is encoded by the nucleic acid, to a particular target site. In particular, a nucleic acid according to the invention can be linked to another nucleic acid which codes for a polypeptide that brings about a translocation or stabilization of the encoded protein or polypeptide on the cell membrane of the host cell or its compartmentalization in particular organelles of this cell.

In a preferred embodiment, a cloned nucleic acid is, according to the invention, present in a vector, with the vector optionally comprising a promoter that controls the expression of the nucleic acid. The term “vector” is used in its most general meaning and comprises any intermediate vehicles for a nucleic acid that make it possible, for example, to insert the nucleic acid into prokaryotic and/or eukaryotic cells and optionally integrate it into a genome. Such vectors are preferably replicated and/or expressed in the cell. An intermediate vehicle can be adapted e.g. for use in electroporation, in microprojectile bombardment, in liposomal administration, in transfer by means of agrobacteria or in insertion via DNA or RNA viruses. Vectors comprise plasmids, phagemids or viral genomes.

Recombinant RNA can be produced according to the invention by in vitro transcription of an appropriate DNA template. It can moreover be modified by stabilizing sequences, capping and polyadenylation.

The term “expression” is used according to the invention in its most general meaning and comprises the production of RNA or of RNA and proteins/polypeptides. It also comprises partial expression of nucleic acids. Moreover, expression can be transient or stable. With reference to RNA, the term “expression” relates in particular to the production of proteins/polypeptides.

“Derivatives” of an expression product, in particular of a protein or polypeptide or of an amino acid sequence in the sense of this invention comprise amino acid insertion variants, amino acid deletion variants and/or amino acid substitution variants.

Amino acid insertion variants comprise amino- and/or carboxy-terminal fusions, and insertions of individual or several amino acids in a particular amino acid sequence. In amino acid sequence variants with an insertion, one or more amino acid residues are inserted at a predetermined location in an amino acid sequence, although random insertion with suitable screening of the resultant product is also possible. Amino acid deletion variants are characterized by the removal of one or more amino acids from the sequence. Amino acid substitution variants are characterized in that at least one residue in the sequence is removed and another residue is inserted in its place. Preferably the modifications are located at positions in the amino acid sequence that are not conserved between homologous proteins or polypeptides. Preferably, amino acids are replaced by others with similar properties, such as hydrophobicity, hydrophilicity, electronegativity, volume of the side chain and the like (conservative substitution). Conservative substitutions relate for example to the replacement of one amino acid by another amino acid, listed below in the same group as the substituted amino acid:

1. small aliphatic, nonpolar or slightly polar residues: Ala, Ser, Thr (Pro, Gly)
2. negatively charged residues and their amides: Asn, Asp, Glu, Gln
3. positively charged residues: His, Arg, Lys
4. large aliphatic, nonpolar residues: Met, Leu, Ile, Val (Cys)
5. large aromatic residues: Phe, Tyr, Trp.

Three residues are put in parentheses on account of their particular role in the protein architecture. Gly is the only residue without a side chain and thus imparts flexibility to the chain. Pro possesses an unusual geometry, which can lead to considerable alteration of the polypeptide structure. Cys can form intra- or intermolecular disulfide bridges with another Cys.

The amino acid variants described above can easily be produced using recombinant DNA manipulation. Techniques for inserting substitution mutations at predetermined sites in DNA, which possesses a known or partially known sequence, are well known and include e.g. M13 mutagenesis. The manipulation of DNA sequences for the production of proteins with substitutions, insertions or deletions is described in detail e.g. in Sambrook et al. (1989).

“Derivatives” of proteins or polypeptides also comprise, according to the invention, single or multiple substitutions, deletions and/or additions of any molecules that are associated with the protein or polypeptide, such as carbohydrates, lipids and/or proteins or polypeptides. Moreover, the term “derivative” also applies to all functional chemical equivalents of proteins or polypeptides.

A portion or fragment of an expression product preferably has, according to the invention, a functional property of the expression product from which it was derived. Said functional properties comprise interaction with antibodies, interaction with other polypeptides or proteins, selective binding of nucleic acids and enzymatic activity.

A portion or a fragment of a nucleic acid preferably relates, according to the invention, to a portion of the nucleic acid that codes for a portion or a fragment of an expression product as defined above.

Various methods can be used for the introduction of nucleic acids into cells according to the invention. Such methods comprise the transfection of nucleic acid-CaPO₄ precipitates, the transfection of nucleic acids that are associated with DEAE, transfection or infection with viruses carrying the nucleic acids of interest, electroporation, liposome-mediated transfection and the like. Directing of the nucleic acid to particular cells is preferred in certain embodiments. In such embodiments a carrier, which is used for delivering a nucleic acid to a cell (e.g. a retrovirus or a liposome), can have a bound directing molecule. For example, a molecule such as an antibody, which is specific to a surface membrane protein on the target cell, or a ligand for a receptor on the target cell, can be incorporated in or bound to the nucleic acid carrier. If introduction of a nucleic acid by liposomes is desired, proteins which bind to a surface membrane protein that is associated with endocytosis can be incorporated in the liposome formulation, to make targeting and/or absorption possible. Such proteins comprise capsid proteins or fragments thereof, which are specific to a particular cell type, antibodies against proteins that are internalized, proteins that guide to an intracellular site, and the like.

The term “complementation analysis” relates, according to the invention, to an investigation of effects that are produced in an organism or a cell when a nucleic acid is introduced into this organism or cell, after a particular gene has been deleted or mutated, so that it is no longer fully functional in its normal role. If a complementary gene or its expression products are introduced into the organism or cell with a deleted or mutated gene, they are preferably capable of reproducing the function of the gene.

The term “microarray” relates, according to the invention, to a device that is used for applying and binding biological material, e.g. nucleic acids, peptides or proteins, at high density. Microarrays consist of or contain a solid carrier substance that permits large quantities of samples to be applied at controlled intervals, so that they can be used for parallel experiments.

The term “high density” is used, according to the invention, in connection with arrays that have a high density of various samples, such as nucleic acid samples, and can thus provide answers to a large number of questions.

The present invention is described in detail by the figures and examples given below, which serve exclusively for illustration and are not to be understood as limiting. A person skilled in the art will be able to access further embodiments on the basis of the description and the examples, and these too are covered by the invention.

### FIGURES

FIG. 1: Representation of the eGFP vectors used—a. pGEM3Z eGFP Tail and b. pSTI eGFP Tail. These vectors were used as starting vectors for the subsequent clonings.

FIG. 2: Western blot for detecting translation of eGFP IVT RNA 24 h after transfection in 786-0 cells. 20 μg eGFP-pGEM3Z Tail IVT RNA was transfected at 200V and 250 μF. Rabbit anti-GFP at a concentration of 1 μg/ml was used as primary antibody. Goat anti-rabbit HRP at a concentration of 0.5 μg/ml was used as secondary antibody. A mouse anti-ERK2 antibody at a concentration of 0.2 μg/ml was used as loading control. Goat anti-mouse HRP was diluted 1:5000 as secondary antibody here.

FIG. 3: Representation of the FACS dot blot 24 h after transfection of 1×10⁷ 786-0 cells with 20 μg eGFP IVT RNA, or after control transfection without RNA.

FIG. 4: Determination of the influence of cell count on electroporation efficiency by determining the mean fluorescence intensity of eGFP. 20 μg eGFP-pGEM3Z Tail IVT RNA was transfected into the stated number of K562 cells. The cells were cultivated after transfection for 24 h. The mean fluorescence intensities were determined using FACS-Calibur.

FIG. 5: Determination of the influence of the electroporation conditions on electroporation efficiency and mean fluorescence intensity. Transfection of 20 μg eGFP IVT RNA in MCF7 cells. The cells were cultivated after transfection for 24 h. The mean fluorescence intensities were determined using FACS-Calibur.

FIG. 6: Determination of the influence of time on transcript and protein quantity after transfection of 2×10⁷ 786-0 cells with 20 μg eGFP IVT RNA or 2dGFP IVT RNA. a. Determination of the relative transcript quantity based on quantitative RT-PCR analysis. Normalization was performed against untransfected cells. To take account of differences in cDNA quality, an 18s-specific PCR was performed and the values were used for normalization. b. Determination of the mean fluorescence intensity of eGFP using the FACS-Calibur.

FIG. 7: Determination of the influence of the amount of RNA on the eGFP protein level 24 h after transfection of 5×10⁶ MCF-7 cells with eGFP IVT RNA. a. Representation of the measurement of fluorescence intensity in the histogram blot in comparison with an untransfected control. b. Representation of the mean fluorescence intensities.

FIG. 8: Quantitative determination of the relative transcript quantity of HIF1-α, LDHA, VEGF and Glut-1 24 h after transfection of 2×10⁷ 786-0 cells by real-time RT-PCR. Representation of relative expression in comparison with an eGFP transfected control.

FIG. 9: Relative transcript quantity of VEGF and LDHA after transfection of 2×10⁷ 786-0 cells with 20 μg VHL IVT RNA—variation over time. The relative transcript quantities were determined in comparison with untransfected cells. Normalization was effected via 18s rRNA.

FIG. 10: Representation of the relative transcript quantities after transfection of 1×10⁷ MCF-7 cells with SYT, SSX2, SYT-SSX1, SYT-SSX2 or eGFP IVT RNA as control. Normalization was effected via HPRT. The relative transcript quantities were determined in comparison with cells transfected with eGFP IVT RNA.

FIG. 11: Representation of the relative transcript quantities of BMP7, EPHA4 and COL5A1 in synovial sarcomas in comparison with normal tissues from mamma, lung, kidney, ovary, testis, liver, lymph nodes, thymus, spleen and adrenal gland. Normalization was effected via HPRT. For determination of the expression of BMP-7 and COL5A1 the sarcoma tested was an osteosarcoma, whereas for investigating EPHA4 a synovial sarcoma was tested, which was not positive for SYT-SSX1 or SYT-SSX2. The diagnosis of the pathology is questionable owing to the lack of translocation.

FIG. 12: Synoptic table of the genes that can be grouped together according to their function and are expressed differentially at least by a factor of 2¹, at a significance criterion of 5%, upon transfection of MCF-7 cells with SSX2, SYT-SSX1 or SYT-SSX2 in comparison with a control transfection with eGFP. The values that are not stated lie in the non-significant regulatory region, i.e. <2¹. The values shown in the table are in each case the exponents to base two; the resultant number expresses the factor of differential expression.

FIG. 13: Representation of the relative transcript quantities in comparison with eGFP-transfected cells determined by real-time RT-PCR. Normalization was effected via HPRT. The standard deviation between the biological triplicates is stated.

### EXAMPLES

### Example 1

### Production of Vectors

The vectors were produced by cloning the respective coding sequence into the pGEM3Z vector (Invitrogen, San Diego, Calif.) or into the pCMV Script vector (Stratagene, Amsterdam, NL) as base vectors.

The pGEM3Z vector has an SP6 promoter before the start codon, starting from which an in vitro transcription is possible. The pCMV Script vector contains a T7 promoter at the same site.

Starting from these base vectors, extensive modifications were introduced, which are necessary for the production of polyadenylated in vitro transcribed (IVT) RNA.

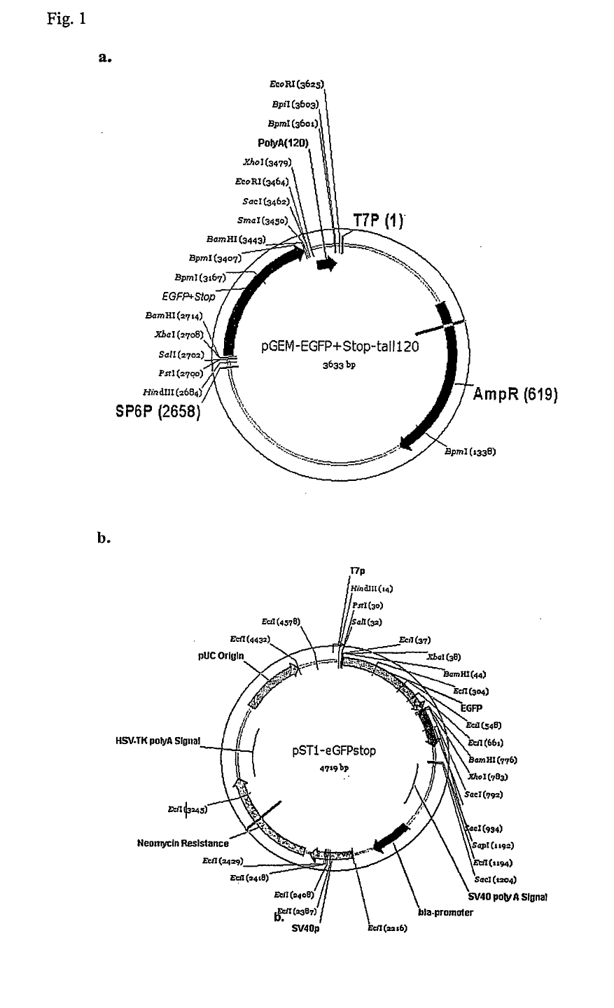

A poly-A stretch of 120 bases was introduced into the pGEM3Z vector via the XhoI cleavage site. Directly after the poly-A stretch, a restriction site was introduced, which permitted linearization. This vector is designated in the following as pGEM3Z Tail vector (FIG. 1a).

A poly-A stretch of 120 base pairs was introduced into the pCMV Script vector behind the SacI cleavage site. This vector is designated in the following as pSTI Tail vector (FIG. 1b).

eGFP or the genes amplified from the respective tissues were ligated after purification and restriction with the respective vectors and transformed in E. coli MRF1⁻. The plasmids obtained were sequenced and the sequence was analyzed.

### Example 2

### Production of IVT RNA

The first step in the production of IVT RNA comprised linearization of the respective plasmids. The restriction enzymes used were BpiI for the pGEM3Z Tail vector and SapI for the pSTI Tail vector. Both cut only once in the vector, namely behind the inserted Tail. Following linearization, the enzyme was inactivated by phenol-chloroform precipitation and removed. For this, an isovolume of a mixture of phenol and chloroform was added to the restriction charge and mixed thoroughly. Brief centrifugation at 10 000×g provided separation into a lower organic phase and an upper aqueous phase, which contains the DNA. The latter was transferred to a new reaction vessel. Then the aqueous phase was mixed with an isovolume of pure chloroform, to remove any phenol residues. After centrifugation, the aqueous phase was removed and precipitated for 2 h by adding two isovolumes of ethanol and 10% v/v 3M sodium acetate pH 4.5 at −20° C.

The DNA was sedimented by centrifugation for 45 min at 10 000×g at 4° C., washed with 70% ethanol for removal of salts, and was taken up in a suitable volume of RNAse-free water. Gel electrophoresis was used to verify that linearization was successful and complete. The concentration of the DNA was determined photometrically at 260 nm. For determination of the purity of the DNA, in addition the optical density was measured at 280 nm to obtain the OD260/280 ratios.

10 μg of linearized DNA was used for the in vitro transcription. For this, 40 μl dNTPs, with ⅘ of the dGTP additionally provided with a Cap-structure, 10 μl 10× buffer, 20 μl dTT and 10 μl of T7 or SP6 polymerase were incubated for 2 h at 37° C. The polymerases bind to their T7 or SP6 recognition sequences, which are located 5′ from the ORF that is to be transcribed, and synthesize the complementary RNA strand.

The IVT RNA was purified with the MegaClear Kit. For this, it was taken up in a binding buffer concentrate, containing the necessary salts for optimal binding of the RNA to the silica membrane. Addition of ethanol removes water from the RNA hydration shell. The mixture was loaded in a silica column and centrifuged at 10 000×g for 2 min. The RNA bound to the column, whereas impurities, e.g. enzyme residues, were washed away. After several washing steps, the purified RNA was eluted. The elution buffer was preheated to 95° C. to make elution more efficient.

Quality control and quantification were performed by gel electrophoresis and by photometry.

### Example 3

### Electroporation of Cells

The principle of electroporation is based on disturbing the transmembrane potential of the cells by a brief current pulse. The alteration of the transmembrane potential by an external stimulus is described by the following equation:

ΔVₘ=fEₑₓₜr cos φ

Vₘ is the transmembrane potential and f is a form factor, which describes the influence of the cell on the extracellular field distribution. fEₑₓ describes the applied electric field, r the cell radius and Φ the angle to the externally applied electric field. Factor f is often given as 1.5, though it depends on many other factors. The electroporation of the cells is successful if the applied electric field exceeds the capacity of the cell membrane, i.e. ΔVₘ is greater than a threshold value ΔVₛ, given as 1 V (Kinosita, K., Jr. and Tsong, T. Y. (1977) Nature 268, 438-441). Since construction of the cell membrane as a bilayer is a feature that is common to eukaryotic cells, this value shows little variation for different cell lines.

Through dielectric breakdown of the transmembrane potential, transiently hydrophilic pores are formed, through which water penetrates into the cell, transporting molecules e.g. nucleic acids into the cells (Weaver, J. C. (1995) Methods Mol. Biol. 55, 3-28; Neumann, E. et al. (1999) Bioelectrochem. Bioenerg. 48, 3-16).

Prior to electroporation, the adherent cells used were cultivated up to semi-confluence, washed with PBS and detached from the cell culture flasks with trypsin. The cells were transferred to medium with 10% FCS (fetal calf serum) and centrifuged for 8 min at 500×g. The pellet was resuspended in the serum free medium X-Vivo and centrifuged again for 8 min at 500×g. This washing operation was carried out two more times in order to remove residues of FCS, which would interfere with subsequent electroporation.

After washing, the cells were adjusted in 250 μl to the desired cell density, transferred to the electroporation cuvettes and placed on ice. After adding the appropriate amount of in vitro transcribed RNA and stirring thoroughly, electroporation was carried out at 200 V and 250 μF. Then the cells were transferred immediately to the adequate nutrient medium and incubated at 37° C. and 5% CO₂.

### Example 4

### Establishment of Experimental Conditions and Characterization of the Experimental System

The eGFP-pGEM3Z Tail expression vector was used for establishing the experimental conditions. The RNA polymerase promoter SP6 located 5′ permits in vitro expression of RNA.

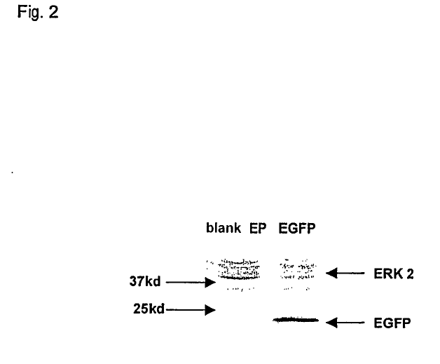

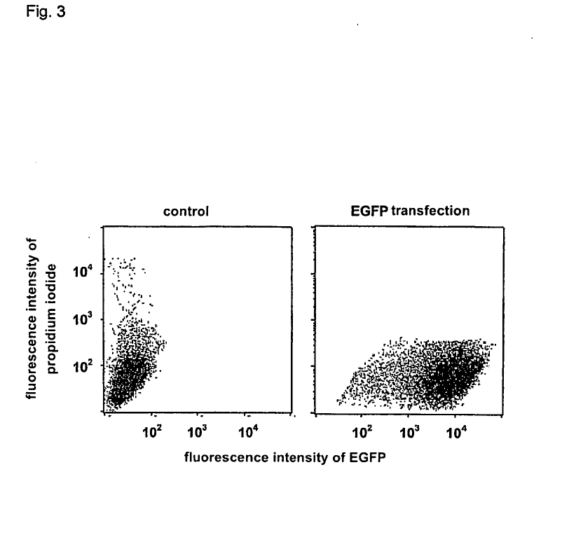

The in vitro transcribed eGFP RNA was used in the following for testing the transfection efficiency of various cell types. Transfection efficiency was determined using the FACS-Calibur via determination of the fluorescence intensity of the eGFP. The amount of RNA in the cells was determined by quantitative RT-PCR. It was shown that not only was it possible to transfect tumor cell lines with an efficiency of >90%, but also primary dendritic cells with an efficiency of 70-80% (FIGS. 2 and 3 and Table 1).

TABLE 1
Transfection efficiencies of various cells determined
after transfection of 5 × 10⁶ cells with 20 μg
eGFP IVT RNA. Measurement of fluorescence intensity
using the FACS-Calibur 24 h after transfection.

| | | % transfected |
|---|---|---|
| Cell line | Cell type | cells |
| HEK | Human embryonic | >90% |
| | kidney cells | |
| K562 | Leukemia cell line | >90% |
| MCF7 | Breast carcinoma | >95% |
| | cell line | |
| 786-0 | Renal cell | >95% |
| | carcinoma cell line | |
| MRC-5 | Lung fibroblasts | >95% |
| Wi-38 | Lung fibroblasts | >90% |
| Monocytes | | 70-80% |
| Dendritic | | 70-80% |
| cells | | |
| Lymphocytes | | 35-45% |

Transfection conditions: electroporation at 200 V and 250 μF

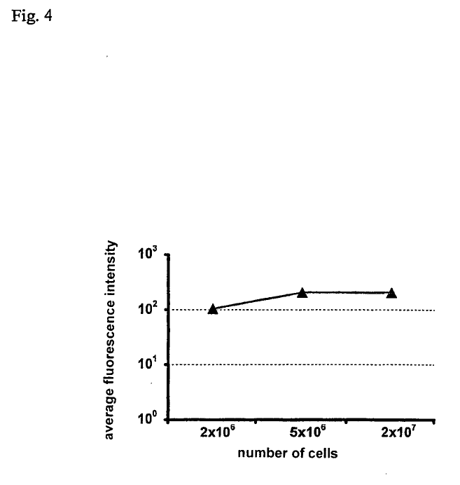

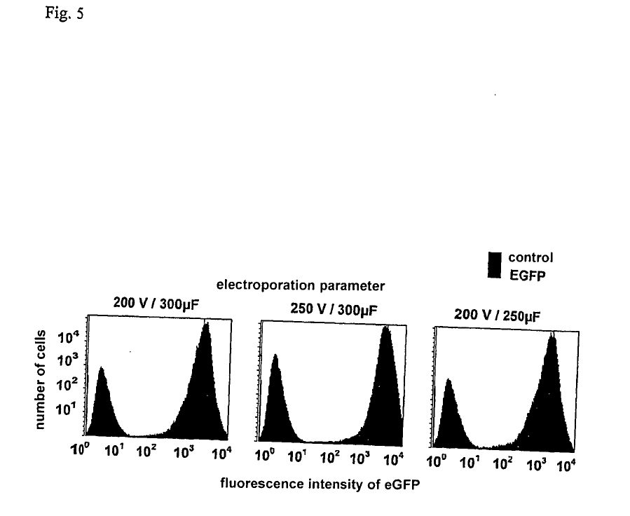

Moreover, an influence of cell count on transfection efficiency could be excluded in a range between 2×10⁶ and 2×10⁷ cells (FIG. 4). The electroporation conditions also do not have a decisive influence on transfection efficiency in the range tested (FIG. 5).

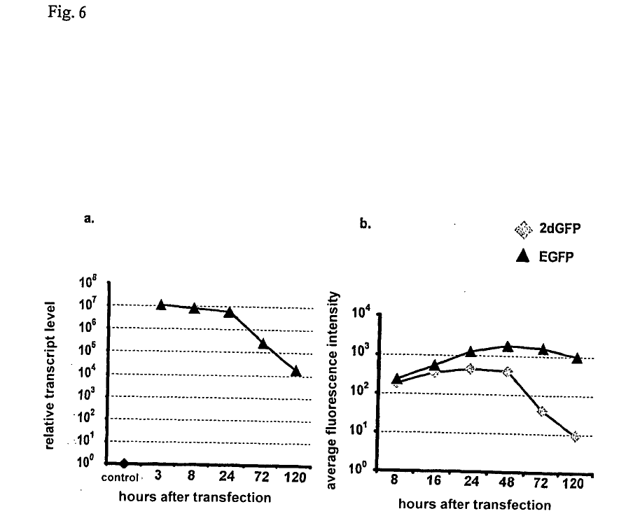

Stability of the RNA was demonstrated over a period of 24 h (FIG. 6a), whereas the protein can be detected in the cells over a period of 48-72 h, depending on its half-life (FIG. 6b).

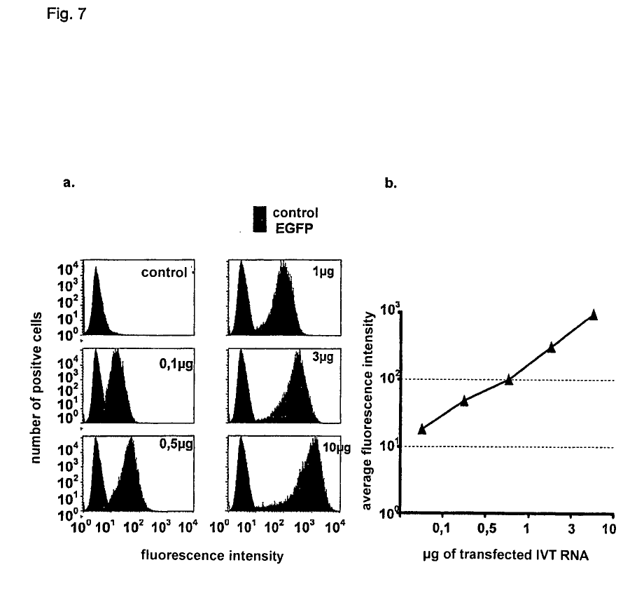

It could be shown that there is a direct dependency between the amount of transfected RNA and the amount of protein available (FIG. 7).

The molecular changes due to the method were determined by means of a cDNA microarray. Molecular changes of proteasome-associated genes, heat-shock genes and apoptosis-associated genes could only be found after 8 h and 24 h (Table 2).

TABLE 2
Number of significantly regulated genes in 2 × 10⁷ 786-0
cells, which were transfected with 20 μg eGFP IVT RNA, in
comparison with untransfected control cells. The changes are
only significant with regulation of >2 or <0.5.

| | Number of | Number of | Number of |
|---|---|---|---|
| Regulatory | regulated genes | regulated genes | regulated genes |
| factor | after 8 h | after 24 h | after 72 h |
| >2 | 10 (0.87%) | 0 | 0 |
| <0.5 | 48 (4.16%) | 15 (1.3%) | 0 |

There was no activation of the interferon system, which has been described after use of siRNA (Elbashir, S. M. et al. (2001) Nature 411, 494-498; Caplen, N. J. et al. (2001) Proc. Natl. Acad. Sci. U.S.A. 98, 9742-9747).

### Example 5

### IVT RNA Transfection of Model Genes

For an even more detailed investigation of the possibilities and limitations of transfection of cells with IVT RNA, various model genes were introduced into cells as heterologously expressed IVT RNA. The well characterized genes HIF1-β (hypoxia inducible factor) and VHL (von-Hippel Lindau) were used as model genes. The coding region of these genes was amplified using cDNA from testis tissue and cloned into the vector system described for eGFP.

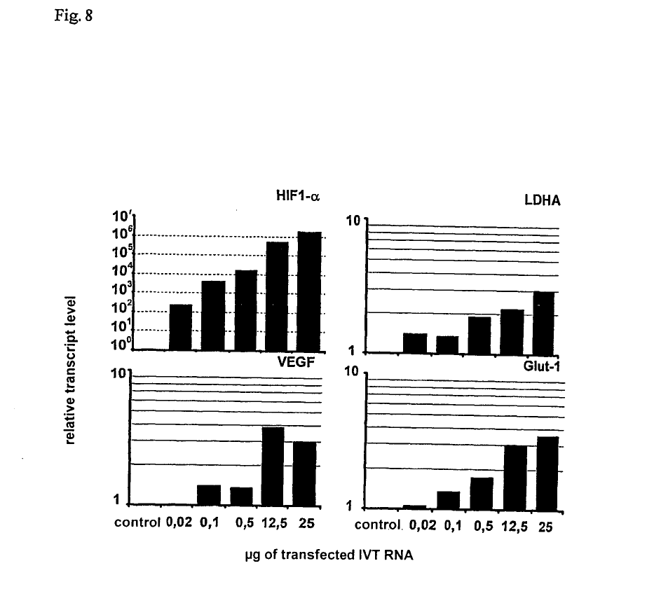

Various target genes are described for HIF1-α and VHL, and their expression was determined after transfection of a deficient cell line. After transfection of the HIF1-α transcription factor, increased expression of the target genes LDHA (lactate dehydrogenase A), VEGF (vascular endothelial growth factor 1 alpha) and Glut-1 (glucose transporter 1) was detected (FIG. 8). The extent of their induction can be titrated on the basis of the amount of transferred HIF1-α IVT RNA. By transfer of the HIF1-α transcription factor, robust regulation of its target genes is possible. Through titration of the amount of IVT RNA used, it is also possible to titrate the transcript quantities of the genes induced by HIF1-α.

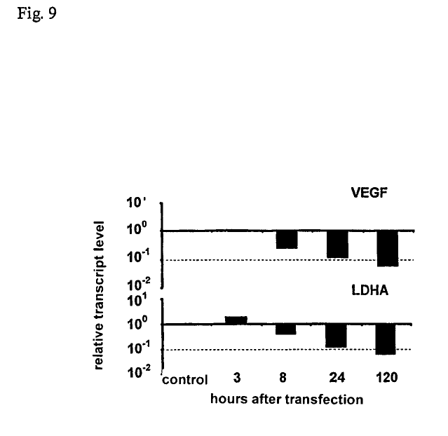

The molecular effects of a transcription factor can be detected almost directly and immediately. Thus, HIF1 binds directly to its consensus sequence in the promoter region of its target genes (Semenza, G. L. (2000) Genes Dev. 14, 1983-1991; Semenza, G. L. (2000) J. Appl. Physiol 88, 1474-1480) and initiates their transcription immediately. To investigate whether indirect and secondary effects can also be manifested by IVT RNA transfer, the pVHL (von-Hippel-Lindau) protein, which precedes the HIF1-α, was also selected as a second model gene. pVHL belongs to the ubiquitin ligase E3 complex and causes degradation of the alpha-subunit of HIF1. Therefore increased expression of VHL leads to a reduced concentration of HIF1 in the cells and an associated reduced expression of HIF1 target genes. The time-dependent decrease in expression of VEGF and LDHA was clearly demonstrated after transfection of cells with VHL (FIG. 9).

### Example 6

### Identification of Hitherto Unknown Functions For Putative Oncogenic Transcripts Using SYT-SSX1 and SYT-SSX2 as an Example

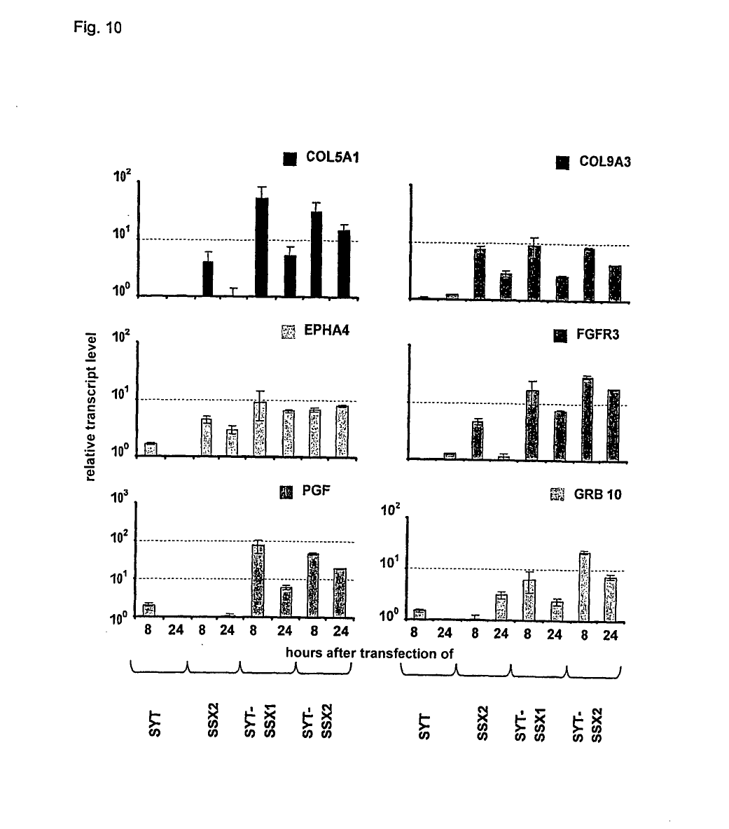

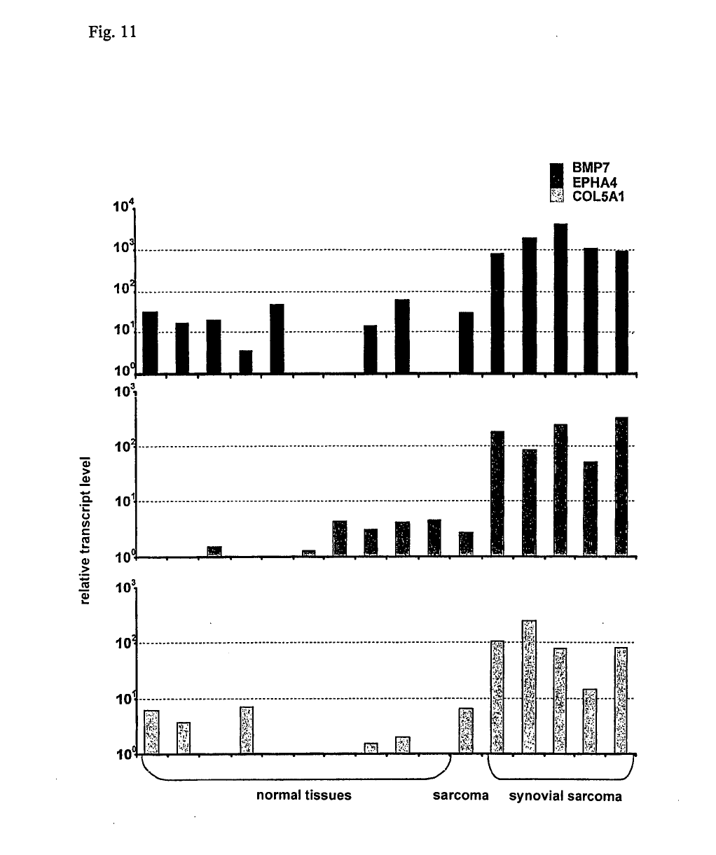

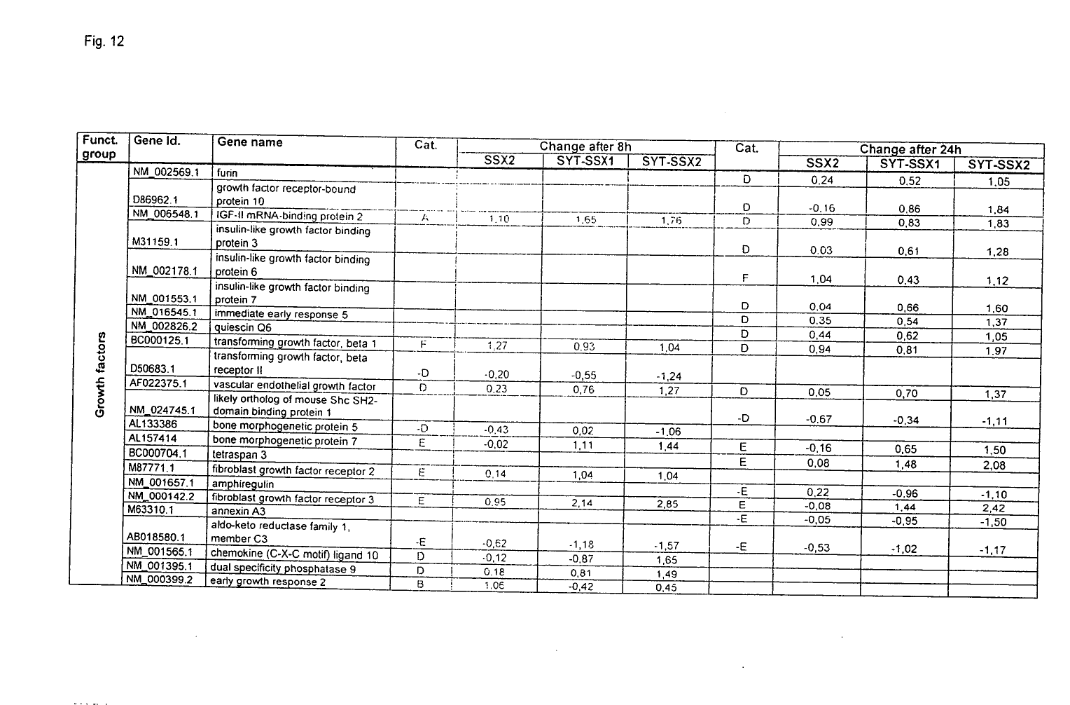

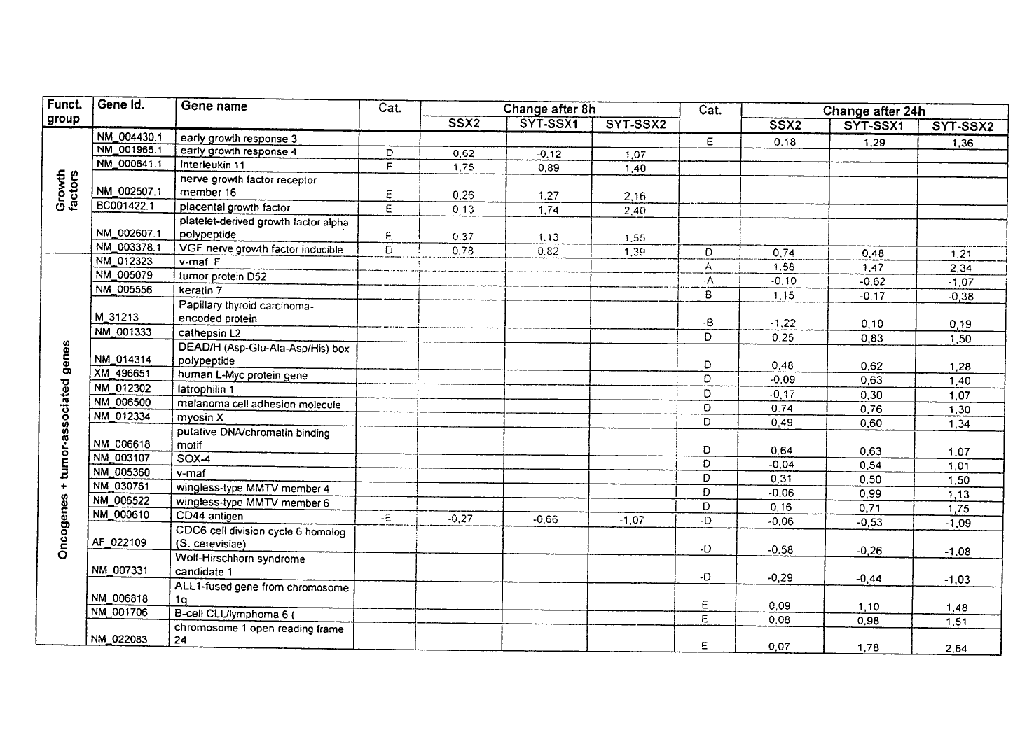

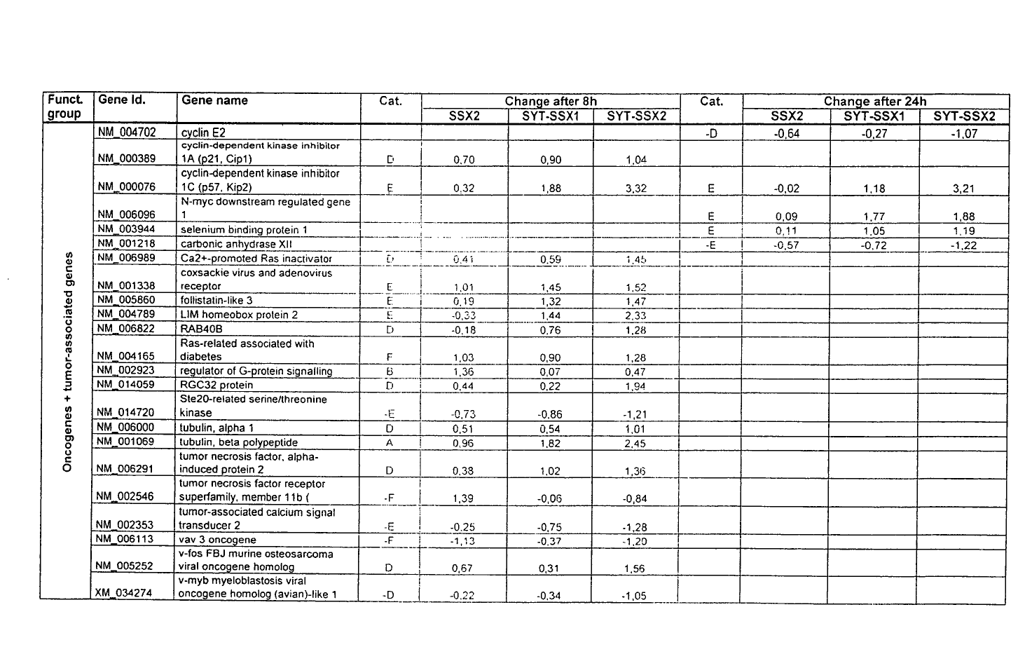

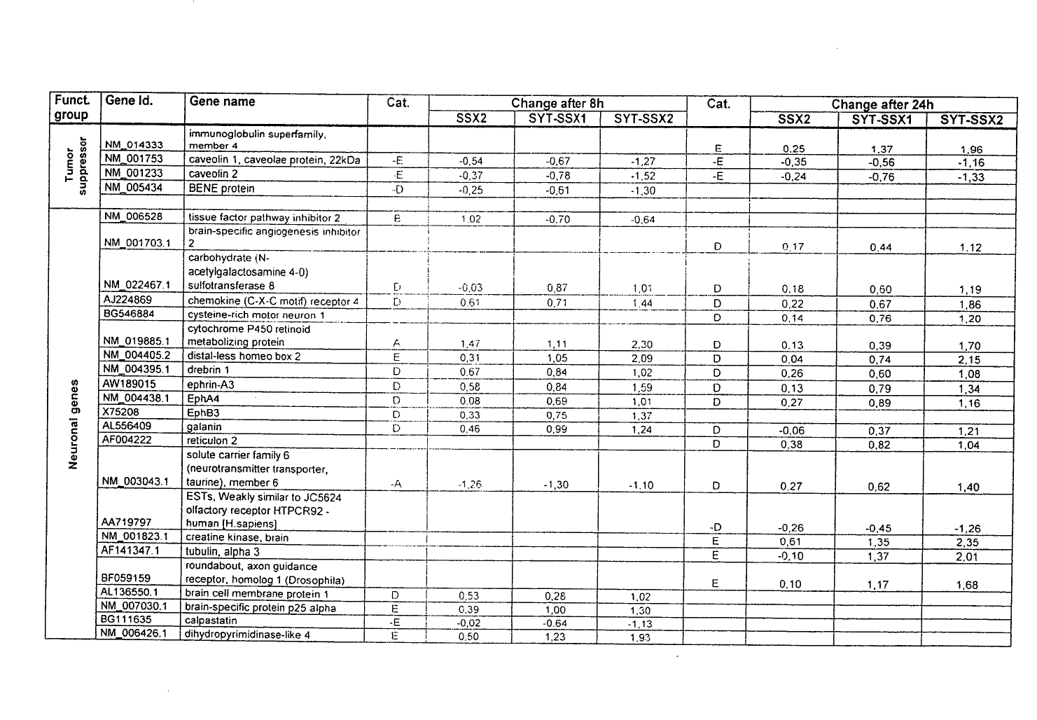

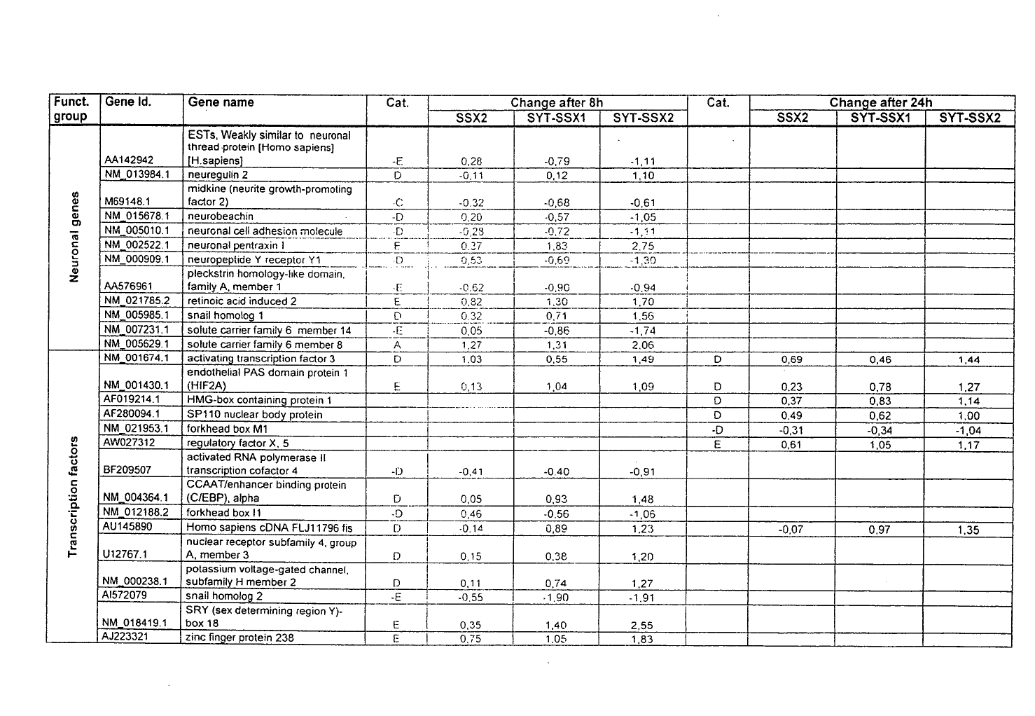

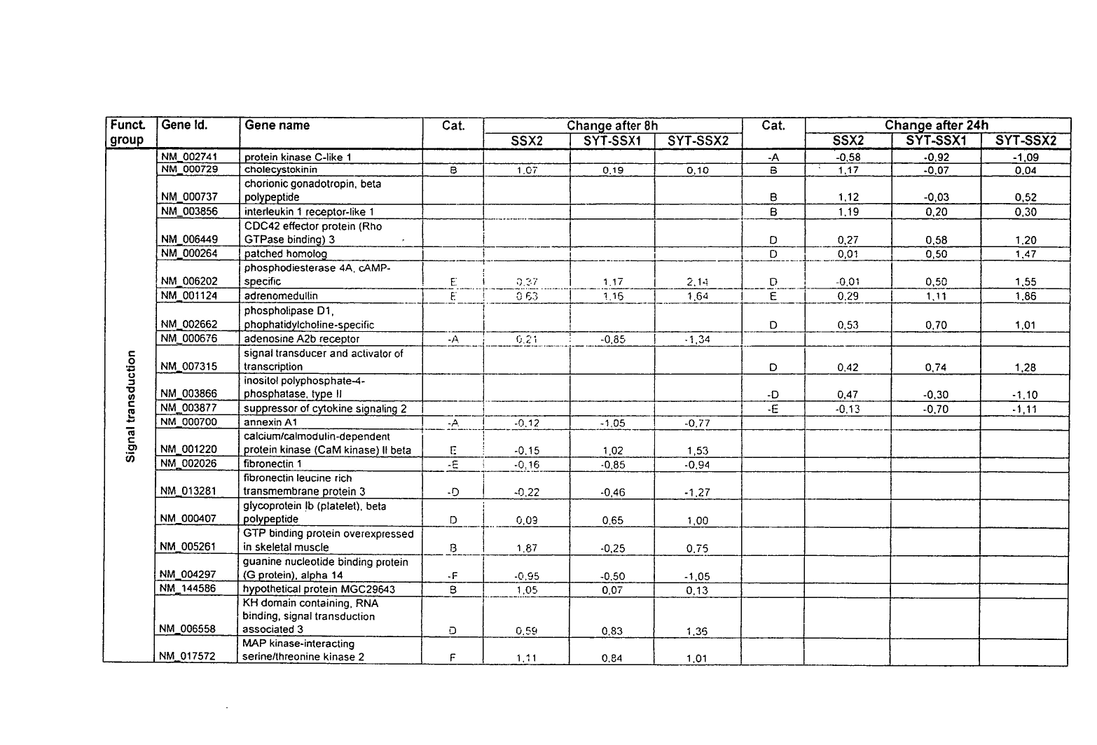

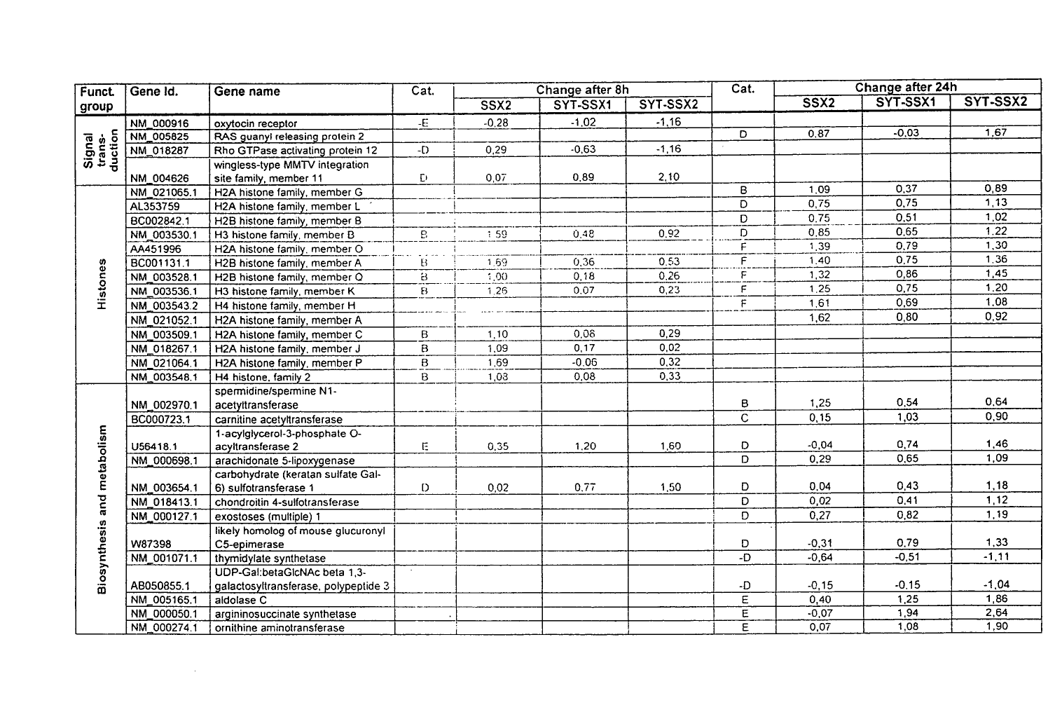

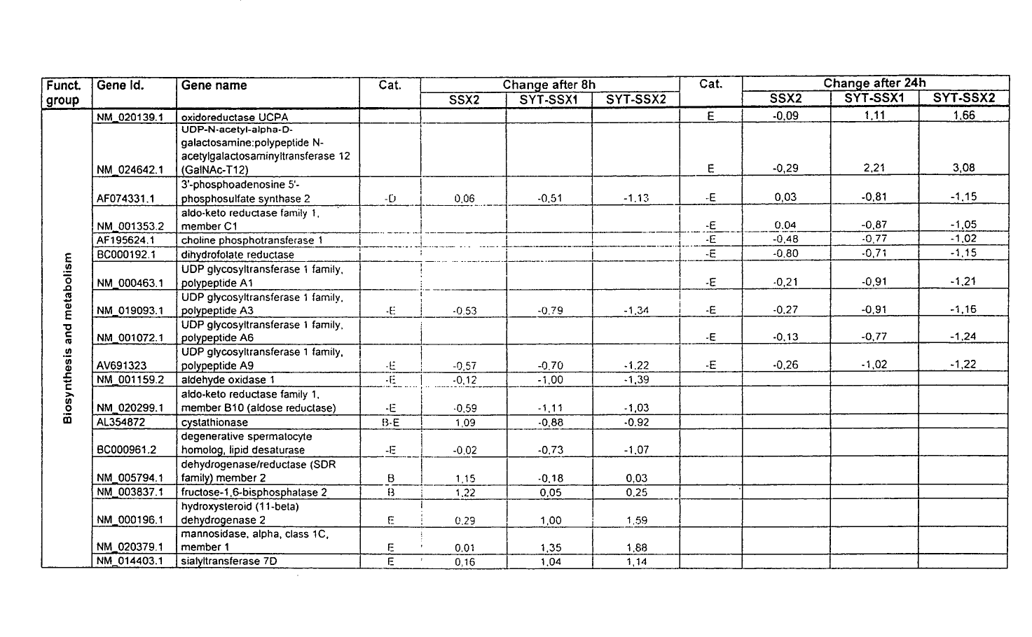

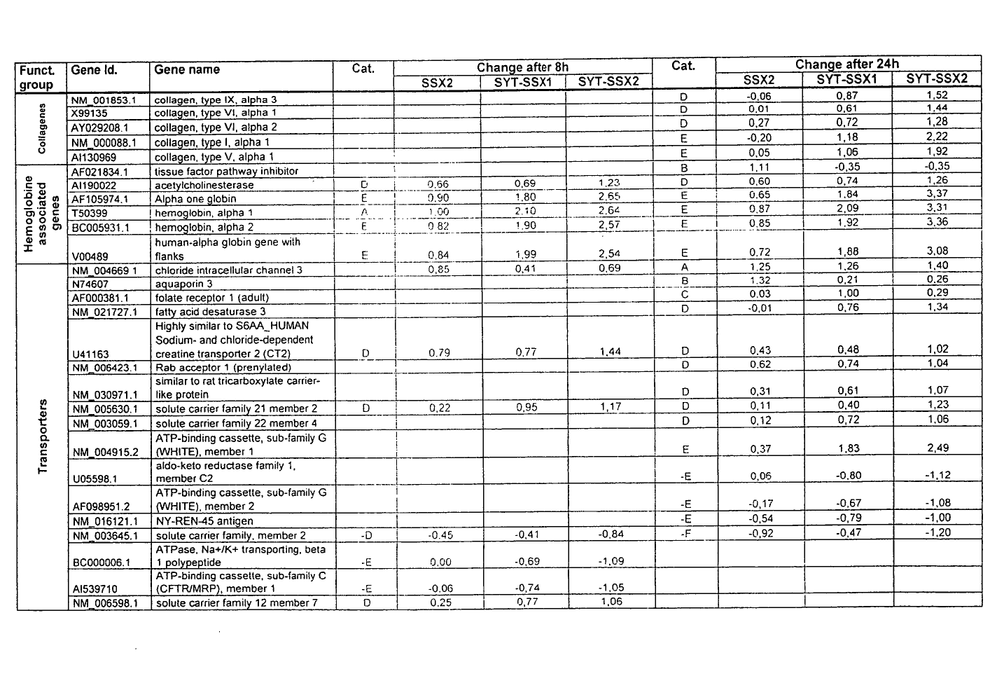

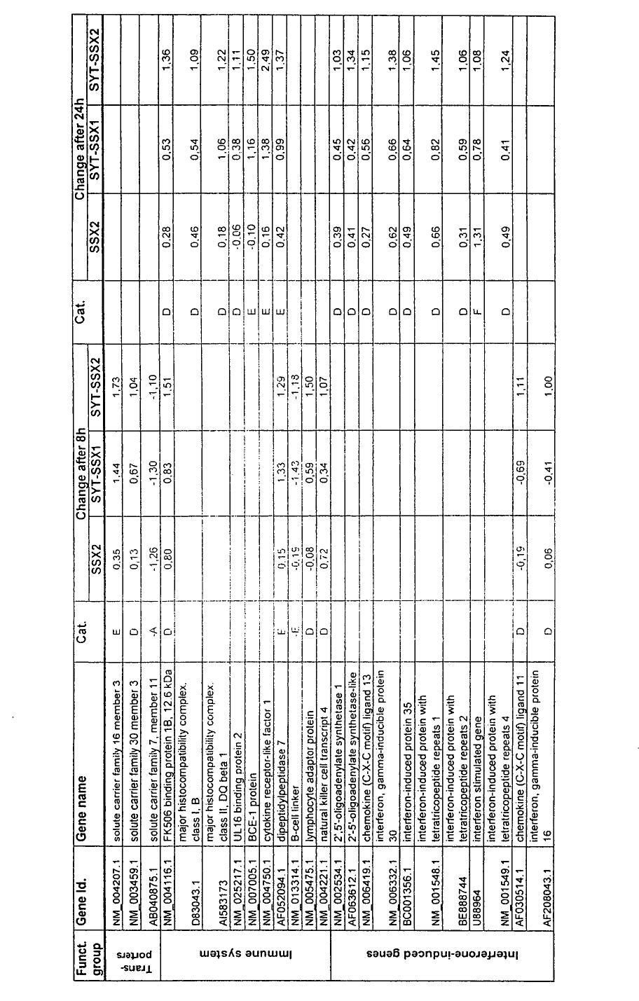

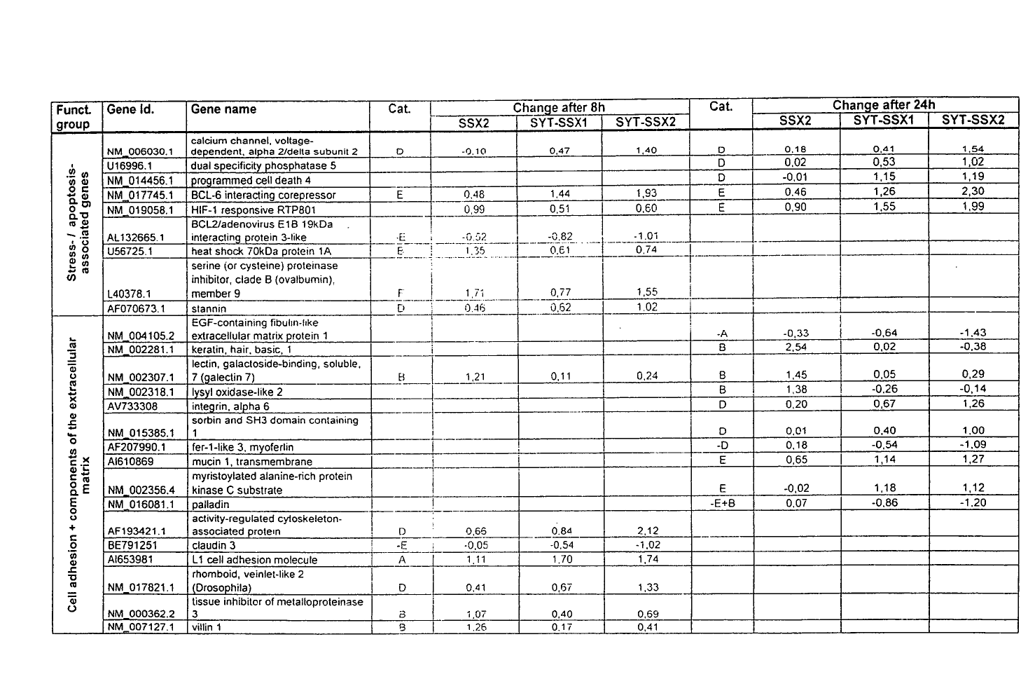

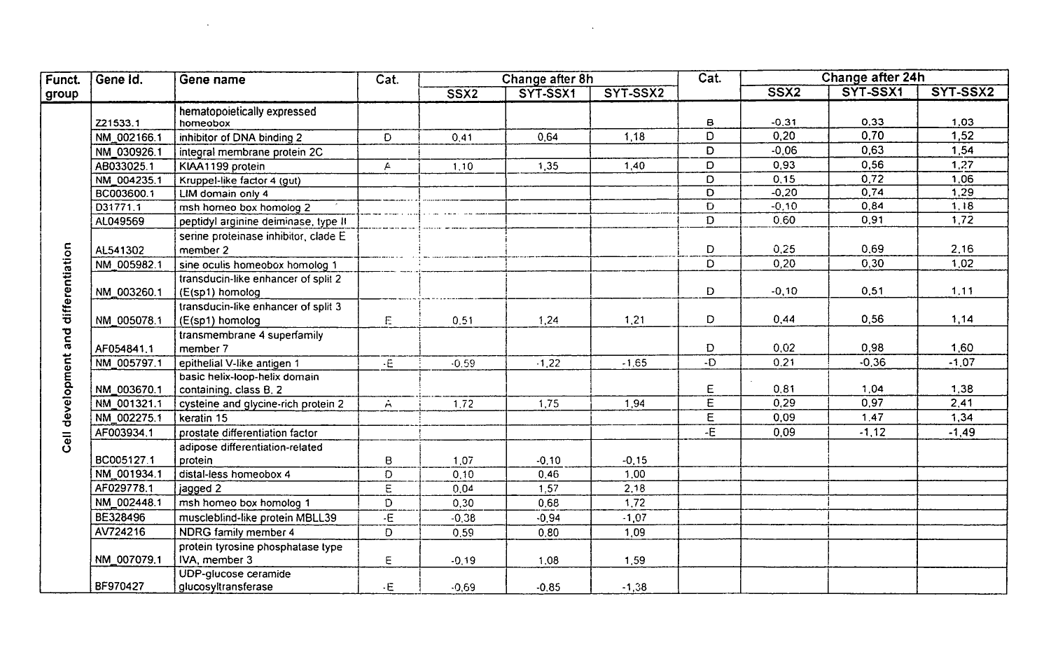

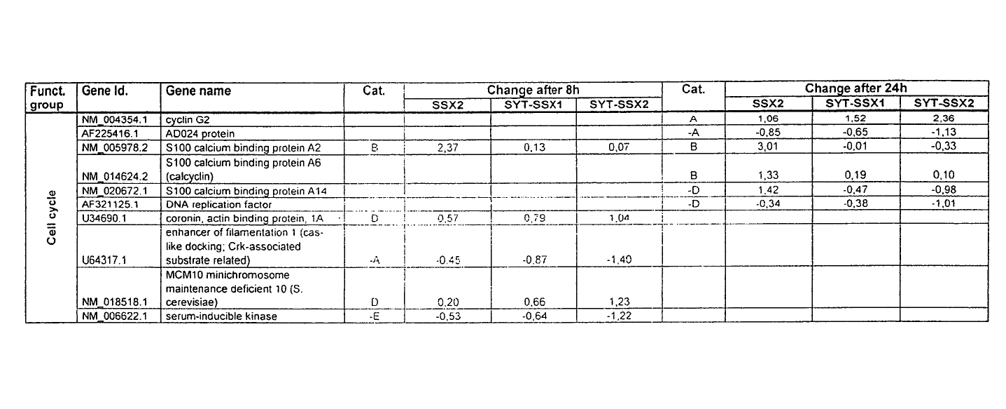

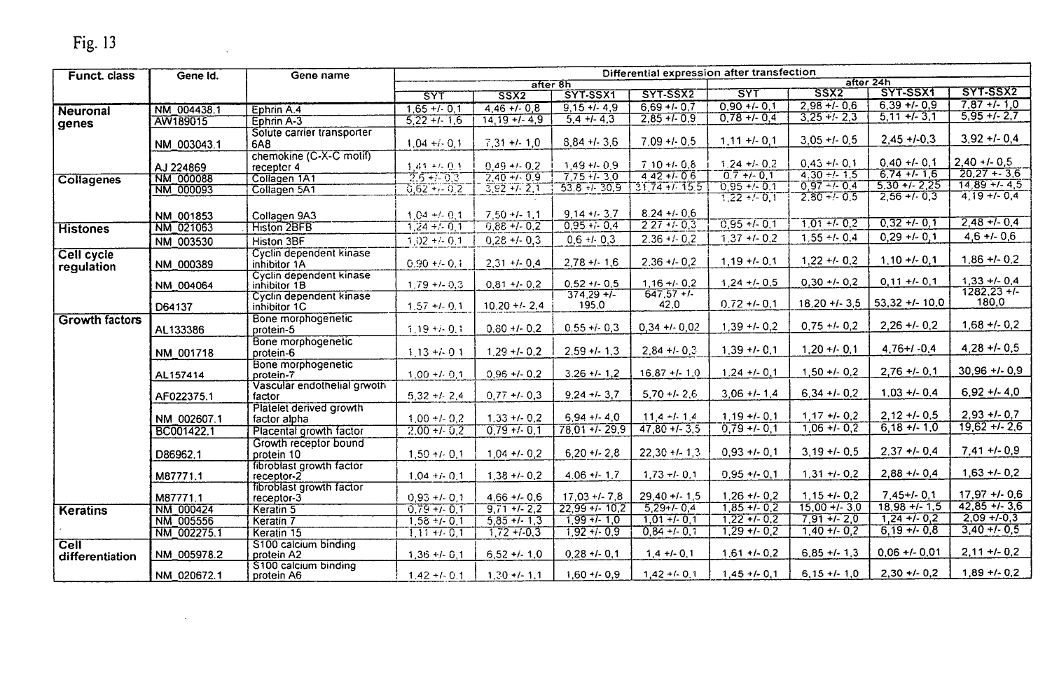

The RNA transfer technique should permit rapid deciphering of the relevance of genes to tumor biology. For verification, the fusion products of the genes SYT, a ubiquitously expressed gene, and SSX1 or SSX2, CG genes, which are only expressed in testis and in tumors, were selected. The reason for choosing these genes was on the one hand the frequency of the translocation t(X;18) (p11.2;q11.2) (Clark, J. et al. (1994) Nat. Genet. 7, 502-508; Crew, A. J. (1995) EMBO J. 14, 2333-2340) in over 90% of synovial sarcomas (Sreekantaiah, C. et al. (1994) Am. J. Pathol. 144, 1121-1134), which suggests relevance for the biology of the tumor and therefore an oncogenic potential. On the other hand the fusion products contain a transcription-activating QPGY domain, so that direct molecular changes are to be expected as a result of translocation. Molecular effects of translocation and their importance in the development and establishment of tumors are still largely unknown. The molecular changes induced by the transfection of SYT-SSX1 and SYT-SSX2 IVT RNA were analyzed using Affymetrix Oligonucleotide Microarrays. After transfection of the cells with SYT-SSX1 and SYT-SSX2, increased expression of numerous genes could be detected. The regulated genes that could be detected after transfection included primarily growth factors, neuronal genes, oncogenes or tumor-associated genes, but also those that play a role in cell cycle regulation or in signal transduction (FIG. 12). The differential expression of some of these genes was verified by quantitative RT-PCR (FIG. 13; FIG. 10). For detecting the functional relevance of these genes in synovial sarcomas, their expression was in addition determined in synovial sarcomas in comparison with normal tissues (FIG. 11).

Using the transfection of SYT-SSX1 and SYT-SSX2 as an example, it could clearly be shown that by means of RNA transfer and subsequent determination of the molecular changes using Affymetrix Oligonucleotide Microarrays it is possible to determine the significance of a gene in carcinogenesis. Direct and indirect target genes of the transfected gene are determined and contribute to our understanding of the gene's function.

In conjunction with the pharmacokinetic characteristics of IVT RNA, this constitutes an excellent basis for deciphering signal transduction cascades. The construction of gene expression profiles at various points of time and their analysis with suitable software should enable us to discover sequentially activated cascades of regulated genes, by describing the course of defined gene clusters. It would thus be possible, with the RNA transfer technique, to elucidate the functional significance of genes that so far are poorly characterized in carcinogenesis. In addition, by subsequent transfer of newly identified target genes it is possible to discover direct and indirect effects of the transfected gene.

1. A method of investigating the function of a nucleic acid or of a nucleic acid expression product in an organism, comprising the steps:
(a) introducing RNA, which has been derived from the nucleic acid or codes for at least a portion of the sequence of the expression product, into a host cell;
(b) investigation of effects on the host cell, which arise as a result of introduction of the RNA into the host cell; and
(c) identification of one or more functions of the nucleic acid or of the expression product based on the effects on the host cell established in step (b).

2. The method as claimed in claim 1, characterized in that the RNA is obtained by cloning the nucleic acid encoding it and in vitro transcription.

3. The method as claimed in claim 2, characterized in that the in vitro transcription is controlled by a T7, T3 or SP6 promoter.

4. The method as claimed in claim 2, characterized in that the nucleic acid is present in combination with a polyadenyl cassette.

5. The method as claimed in claim 1, characterized in that the RNA has a polyadenylation of a defined length.

6. The method as claimed in claim 1, characterized in that the RNA has a Cap structure.

7. The method as claimed in claim 1, characterized in that the introduction of the RNA into the host cell brings about expression of the expression product encoded by the RNA.

8. The method as claimed in claim 7, characterized in that the expression product is selected from the group consisting of peptides, protein, enzymes, hormones and antibodies.

9. The method as claimed in claim 1, characterized in that the investigation of effects on the host cell comprises an investigation of phenotypic and/or biochemical characteristics of the host cell.

10. The method as claimed in claim 1, characterized in that the investigation of effects on the host cell comprises an investigation of the protein expression profile (proteome analysis) of the host cell.

11. The method as claimed in claim 1, characterized in that the investigation of effects on the host cell comprises an investigation of the RNA expression profile (transcriptome analysis) of the host cell.

12. The method as claimed in claim 1, characterized in that the investigation of effects on the host cell comprises an investigation of the host cell's metabolism.

13. The method as claimed in claim 12, characterized in that the investigation of metabolism comprises an investigation of the accumulation of substrates or products of enzymatic reactions.

14. The method as claimed in claim 1, characterized in that the investigation of effects on the host cell comprises an investigation of gene regulation.

15. The method as claimed in claim 1, characterized in that the effects on the host cell are investigated in comparison with a cell lacking the introduced RNA.

16. The method as claimed in claim 1, characterized in that the investigation of effects on the host cell comprises a complementation analysis.

17. The method as claimed in claim 1, characterized in that the host cell is a prokaryotic or eukaryotic cell.

18. The method as claimed in claim 1, characterized in that the introduction of RNA into the host cell is carried out by electroporation or by means of liposomes.

19. The method as claimed in claim 1, characterized in that the investigation of effects on the host cell is carried out by means of a high-density microarray arrangement.

20. The method as claimed in claim 3, characterized in that the nucleic acid is present in combination with a polyadenyl cassette.


SEQUENCE LISTING (1 sequence)

SEQ ID NO: 1 - RNA, 500 nt, Artificial Sequence
  SYNTHETIC POLYADENYLATION SEQUENCE
aaaaaaaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn